(19) United States
(12) Patent Application Publication
(10) Pub. No.: US 2004/0204732 A1
(43) Pub. Date: Oct. 14, 2004

(54) Tubular microsurgery cutting apparatus and method

(76) Inventor: Semeyn Muchnik, San Francisco, CA (US)

Correspondence Address: John W. Carpenter, Carpenter & Kulas, LLP, Suite 109, 1900 Embarcadero Road, Palo Alto, CA, 94303, US

(21) Appl. No.: 10/412,402

(22) Filed: Apr. 12, 2003

(51) Int. Cl. A61B 17/32

(52) U.S. Cl. 606/171; 606/167; 606/170

(57) ABSTRACT

A vitreous removing apparatus for intraocular surgery comprising an outer tube and an inner tube concentric with the outer tube. The outer tube includes a tubular body and a tubular cutting-zone section. The tubular body has a larger internal diameter than an internal diameter of the cutting-zone section. The inner tube has an inner body and a distal cutting end. The inner body has an outer diameter which is essentially equal to an outer diameter of the distal cutting end. A method for performing intraocular surgery comprising reciprocating a distal cutting end of an inner tubular body within a tubular cutting-zone section of an outer tubular body.

### BACKGROUND OF THE INVENTION

1. Field of the Invention

This invention relates generally to a surgical instrument for cutting and removing biological tissue. More particularly, embodiments of the present invention provide a tubular microsurgery cutting apparatus and method for intraocular surgeries, such as vitrectomy, requiring removal of vitreous or pathologic membranes from the interior of an eye.

2. Description of the Prior Art

The vitreous humor fills a large portion of the interior of the eye behind the lens. It is a relatively tough tissue composed of rather complex substance including long protein molecules joined by patches of secondary protein molecules. As known in the art, a vitrectomy involves removing at least part of vitreous humor and replacing the removed vitreous humor with a saline composition of matter.

It is axiomatic that the intricate procedures of a vitrectomy demand high precision tools that are sufficiently diminutive to enable adept surgical maneuvering within the interior of a human eye. Among the instruments used in intraocular surgeries are those having a small outer tube with an opening in proximity to one end and a concentric inner tube member which provides a cutting edge. Representative concentric cutting tube assemblies are those disclosed in U.S. Pat. No. 4,819,635 to Shapiro and U.S. Pat. No. 5,843,111 to Vijfvinkel. The concentric cutting tube assemblies in both U.S. Pat. No. 4,819,635 to Shapiro and U.S. Pat. No. 5,843,111 to Vijfvinkel have an inner tube with a portion having a larger diameter than the remaining portion of the inner tube. Such inner tubes in combination with extended traveling and cutting areas when concentrically moving within the associated outer tubes suffer from lack of efficiencies.

Therefore, what is needed and what has been invented is a high precision surgical instrument suitable for intraocular surgery and providing more efficient and longer-lasting cutting and operational capabilities than those currently existing. What is further needed and what has been invented is an improved tubular microsurgery cutting apparatus and method for effecting intricate surgery, particularly for performing opthalmic surgery in a more efficient, improved manner.

### SUMMARY OF EMBODIMENTS OF THE INVENTION

Embodiments of the present invention provide a vitreous removing apparatus for intraocular surgery comprising an outer tube including a tubular body and a tubular cutting-zone section. The tubular body has an internal diameter which is larger than an internal diameter of the cutting-zone section. The vitreous removing apparatus further comprises an inner tube concentric with the outer tube and having an inner body and a distal cutting end. The inner body has an outer diameter which is essentially equal to an outer diameter of the distal cutting end. Thus, the outer diameter of the inner body and the distal cutting end is generally a uniform continuous outside diameter.

Embodiments of the present invention provide a method for performing intraocular surgery (e.g., removing a vitreous base adherent to a retinal surface) comprising reciprocating a distal cutting end of an inner tubular body within a tubular cutting-zone section of an outer tubular body. The inner tubular body has a non-flanged or non-expanded distal cutting end. The tubular cutting-zone section has an internal diameter which is smaller than at least one other internal diameter of the outer tubular body.

These provisions together with the various ancillary provisions and features which will become apparent to those artisans possessing skill in the art as the following description proceeds are attained by devices, assemblies, systems and methods of embodiments of the present invention, various embodiments thereof being shown with reference to the accompanying drawings, by way of example only, wherein:

### BRIEF DESCRIPTION OF THE DRAWINGS

FIG. 1 is a cross-sectional assembly view of an embodiment of the tubular microsurgery cutting apparatus of the present invention.

FIG. 2 is a side elevational view illustrating the inner tube concentrically disposed within the outer tube.

FIG. 3A is a side elevational view of the outside tubular cutter.

FIG. 3B is a top plan view taken in direction of the arrows and along the plane of line 3B-3B in FIG. 3A.

FIG. 4 is a side elevational view of the inside tubular cutter with the coating being sectionalized.

FIG. 5 is a graphical illustration of an embodiment of the tubular microsurgery cutting apparatus inserted into an eye for intraocular surgery.

FIG. 6 is a partial vertical view of the inner tubular cutter concentrically disposed within the outer tubular cutter and postured for being driven forward in a reciprocating manner to cut any tissue positioned in an opening in the outer tubular cutter.

FIG. 7 is the partial vertical view of FIG. 6 after the inner tubular cutter was driven forward for cutting tissue positioned in the opening of the outer tubular cutter.

FIG. 8 is the partial vertical view of FIG. 7 after the inner tubular cutter was reciprocated back from the forward position illustrated in FIG. 7.

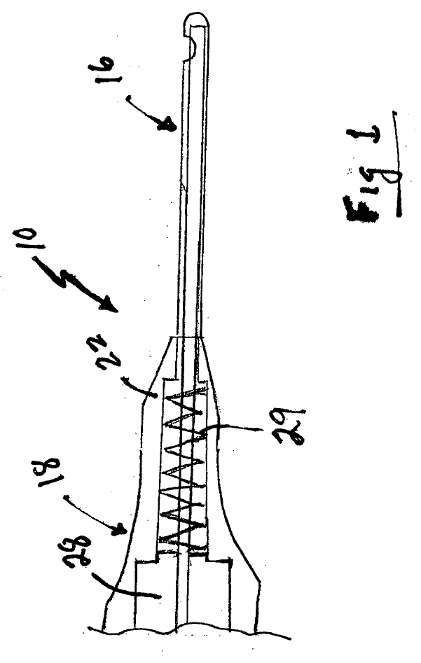

### DETAILED DESCRIPTION OF EMBODIMENTS OF THE INVENTION

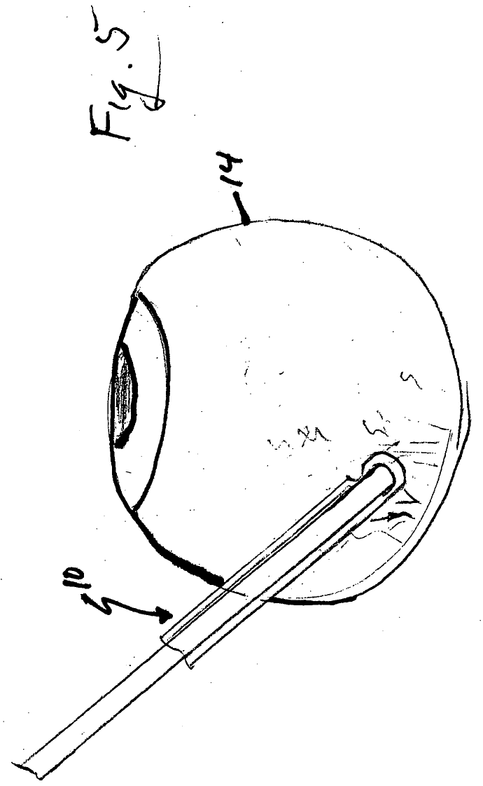

Referring in detail now to the drawings for various embodiments of the present invention and wherein similar parts of the invention are identified by like reference numerals, there is an assembly, generally illustrated as 10, for conducting microsurgery, such as in the eye 14 as illustrated in FIG. 5.

The assembly 10 includes an elongated tubular cutting end 16 and a stationary or hand held end 18. The cutting end 16 has an outer tubular member 20 with an open end 20a and a closed end 20b. The open end 20a of the outer tubular member 20 is affixed to a housing 22 of the hand held end 18. The cutting end 16 also has an inner tubular sleeve 24 having an open end 24a and a cutting end 24b that is adapted to reciprocate within the outer tubular member 20. Those artisans possessing ordinary skill in the art with respect to the assembly 10 frequently refer to the elongated tubular cutting end 16 as the probe, with the outer tubular member 20 representing a needle portion of the assembly 10 and the inner tubular sleeve 24 representing the cutter portion of the assembly 10.

The open end 24a of the inner tubular sleeve 24 is fixed to a reciprocatable piston 28 within the housing 22 of the assembly 10. The cutting end 24b is positioned within the outer tubular member 20 in proximity to the closed end 20b associated therewith. The housing 22, as illustrated for an embodiment of the present invention, is cylindrical and substantially air tight and includes an end cap (not shown) for enclosing the cylindrical piston 28 for reciprocating movement. A source of pulsing air pressure or other driving force is supplied through the end cap to force the piston 28 toward the cutting end 16 of the assembly 10 and against a spring 29 that biases the piston 28 away from the cutting end 16 and toward the end opposed to the cutting end 16. Since the inner tubular sleeve 24 is fixed to the piston 28, the inner tubular sleeve 24 reciprocates with the piston 28 while disposed generally concentric within the outer tubular member 20. The inner tubular sleeve 24 is illustrated as a generally continuous tube extending beyond the end of the driving end 18 of the assembly 10, and is connected to an external vacuum source (not shown) for drawing material (i.e., severed biological material) through the inner tubular sleeve 24 from the cutting end 16 toward the driving end 18.

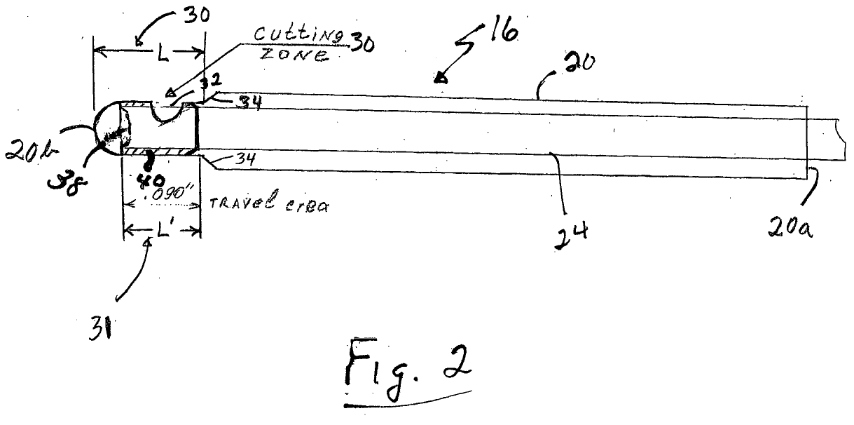

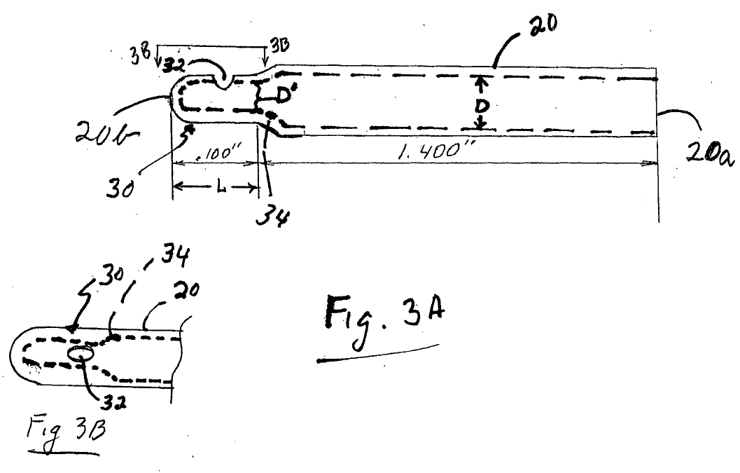

Referring now to FIGS. 2, 3A, 3B and 4, there is seen as indicated the cutting end 16 having the inner tubular sleeve 24 concentrically disposed within the outer tubular member 20. The outer tubular member 20 has a cutout opening defining an entry port 32 for receiving biological material which is to be severed by a cutting edge of the inner tubular sleeve 24. The outer tubular member 20 also includes a discontinuous internal diameter D. More specifically, internal diameter D of the main body of the outer tubular member 20 diminishes, tapers or converges into a cutting zone 30 section of the outer tubular member 20 having an internal diameter D′ which is smaller or less than internal diameter D. The inside cylindrical surface having diameter D is interconnected to or coupled to the inside cylindrical surface having diameter D′ by funnel or converging section 34. Preferably, the length L (see FIGS. 2 and 3A) of cutting zone 30 is less than about 0.30 inches, more preferably less than about 0.20 inches, most preferably about 0.10 inches. The cutting zone 30 of the outer tubular member 20 includes a cutter-traveling area or zone 31 where the cutting head or section (identified as “36” below) of the inner tubular sleeve 24 travels. The length L′ (see FIG. 2) of cutter-traveling area or zone 31 is slightly less than the length L of the cutting zone 30. Thus, the length L′ is less than about 0.30 inches, more preferably less than about 0.20 inches, most preferably less than about 0.10 inches, such as about 0.09 inches.

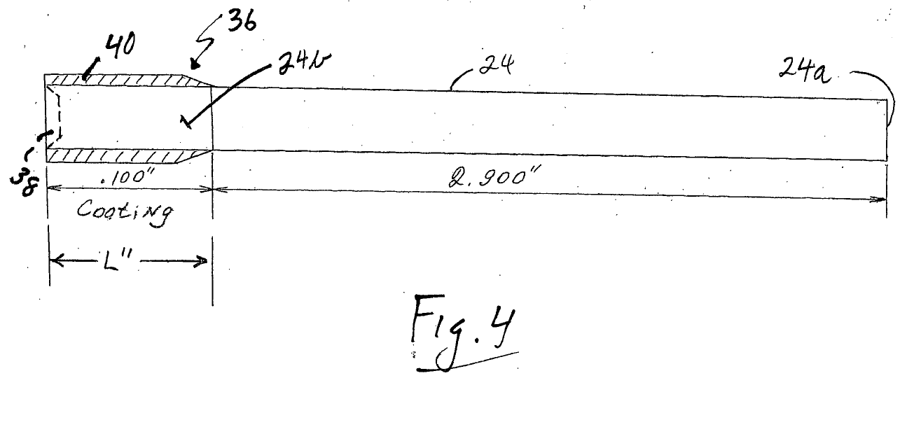

The inner tubular sleeve 24 has a generally uniform exterior or outside diameter and a generally uniform interior or internal diameter. The inner tubular sleeve 24 has a cutting section, generally illustrated as 36, including a cutting tip or surface at 38. The cutting section 36 is covered with a suitable coating 40 (e.g., a chemical coating, an electrolyzed surface, a plate of harder material selected for its hardness and/or wear resistance, or any of the like) to assist in forming the cutting tip or surface 38 and to facilitate the reciprocating motion in a reduced-friction contacting manner. The coating 40 preferably has a length L″ (see FIG. 4) that approximates the length L of the cutting zone 30, which is slightly longer than the length L′ of the cutter-traveling area or zone 31. Preferably, the length L″ (see FIGS. 2 and 3A) of the coating 40 is less than about 0.30 inches, more preferably less than about 0.20 inches, most preferably about 0.10 inches.

The cutter-traveling area or zone 31 where the cutting head or section 36 of the inner tubular sleeve 24 travels represents the axial reciprocation distance of the inner tubular sleeve 24 within the outer tubular member 20. This provides for a more efficient, reduced friction reciprocating cutting movement, particularly since the cutting distance or length L′ is less that about 0.100 inches. The combination of a stationary outer tubular member 20 (including its associated internal diminished diameter D′ of the cutting zone 30 section) with a moving inner tubular sleeve 24 (including its associated uniform outside diameter, as opposed to having a flanged distal end or expanded outside diameter section as illustrated in U.S. Pat. Nos. 4,819,635 and 5,843,111), provides a more efficient cutting assembly than those currently existing.

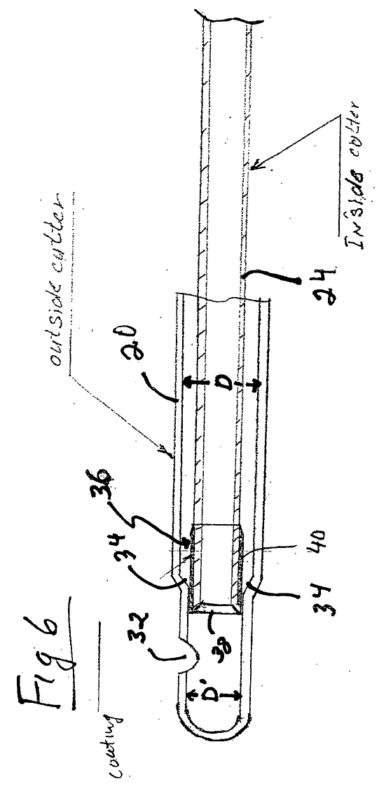

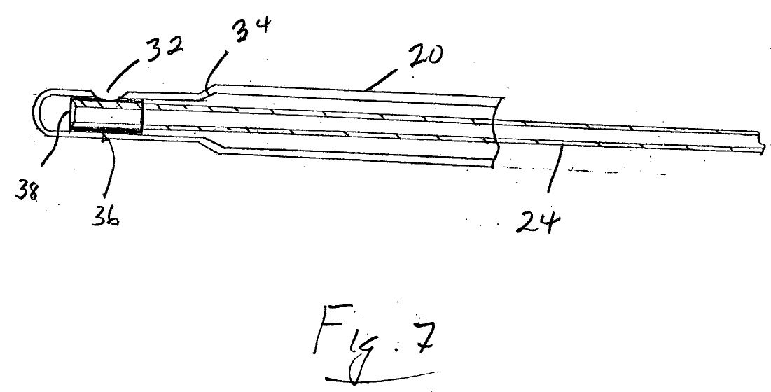

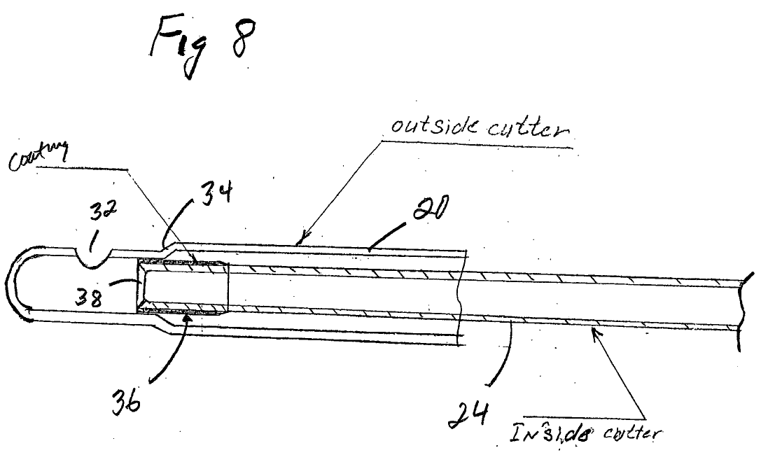

Referring now to FIGS. 6-8 there is seen in FIG. 6 a partial vertical sectional view of the inner tubular sleeve 24 concentrically disposed within the outer tubular member 20 and postured for being driven forward in a reciprocating manner to cut any tissue positioned in the entry port 32 of the outer tubular member 20. FIG. 7 is a partial vertical sectional view illustrating the position of the inner tubular sleeve 24 after being driven forward for cutting biological tissue positioned in the entry port 32 of the outer tubular member 20. FIG. 8 is a partial vertical sectional view illustrating the position of the inner tubular sleeve 24 after the inner tubular member 20 was reciprocated back from the forward position illustrated in FIG. 7.

Embodiments of the assembly 10 are more productive than conventional microsurgery tubular cutting devices because the cutting area or zone of the whole device or assembly is located within the first 0.100 inch. The distance between the inside diameter of the outer tubular member 20 and the outside diameter of the inner tubular sleeve 24 may be any suitable distance to produce a snug, tight fit for an efficient, productive cutting operation. Preferably, the distance between the inside diameter of the outer tubular member 20 and the outside diameter of the inner tubular sleeve 24 is no more than the thickness of the coating 40, more preferably less than about 0.0004 inches, most preferably about 0.0003 inches or less.

As indicated, the type of coating 40 (e.g., an electrolyzed surface) selected will produce a smooth working finish for reducing and/or minimizing friction during the reciprocating cutting operation. Thus, the geometric form or shape of the cutting tip 38 and the body of the inner tubular sleeve 24 (e.g., the cutting head or section 36 of the inner tubular sleeve 24) produces a firm and smooth working contact with the outer tubular member 20 in the cutter-traveling area or zone 31.

Because the inner tubular sleeve 24 has no expanded or flanged end supporting a cutting section (i.e., a flanged distal end or expanded outside diameter section as illustrated in U.S. Pat. Nos. 4,819,635 and 5,843,111) and is generally uniform in diameter through out its stock and/or main body (excepting the miniscule thickness of the coating 40) there is no bending of the inner tubular sleeve 24 in the reciprocating-piston driving operation. In conventional microsurgery tubular devices, such as those disclosed in U.S. Pat. Nos. 4,819,635 and 5,843,111, the body of an inner tubular sleeve between a flanged cutting head and a reciprocating piston within a hand held section will bend, especially with any misalignment off of or away from tubular concentricity, causing the flanged cutting head to abrasive contact and/or cut into the internal surface of the outer tubular member. Such misalignment and/or bending motion of the inner tubular sleeve produces metal dust or chips and shortens the operational life of the conventional microsurgery tubular devices.

The generally uniform diameter of the inner tubular sleeve 24 (i.e., the diameter of the main body and of the cutting head or section 36 of the inner tubular sleeve 24 is the same and/or is a common diameter) in combination with the internal diminished diameter D′ cutting zone 30 section of the stationary outer tubular member 20 produces an essentially dust-free reciprocating-cutting operation. Friction between the reciprocating cutting head or section 36 of the inner tubular sleeve 24 with the internal diminished diameter D′ cutting zone 30 section of the stationary outer tubular member 20 is essentially about non-existent. Therefore, the assembly 10 of the present invention can operate for longer periods of time at high speeds and can be reused for future surgeries. The operational speed of the assembly 10 ranges from about 600 to about 1600 cuts per minute, preferably from about 800 to about 1600 cuts per minute, more preferably from about 1000 to about 1600 cuts per minute, and most preferably from about 1200 cuts per minute to about 1600 cuts per minute (e.g., about 1200 cuts per minute).

The foregoing description of illustrated embodiments of the present invention, including what is described in the Abstract of the Disclosure, is not intended to be exhaustive or to limit the invention to the precise forms disclosed herein. While specific embodiments of, and examples for, the invention are described herein for illustrative purposes only, various equivalent modifications are possible within the spirit and scope of the present invention, as those skilled in the relevant art will recognize and appreciate. As indicated, these modifications may be made to the present invention in light of the foregoing description of illustrated embodiments of the present invention and are to be included within the spirit and scope of the present invention.

Thus, while the present invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the present invention. It is intended that the invention not be limited to the particular terms used in following claims and/or to the particular embodiment disclosed as the best mode contemplated for carrying out this invention, but that the invention will include any and all embodiments and equivalents falling within the scope of the appended claims.

What is claimed is:

1. A vitreous removing apparatus for intraocular surgery comprising:
an outer tube including a tubular body and a tubular cutting-zone section, said tubular body having a larger internal diameter than an internal diameter of the cutting-zone section; and
an inner tube concentric with the outer tube and having an inner body and a distal cutting end, said inner body having an outer diameter which is essentially equal to an outer diameter of the distal cutting end.

2. The vitreous removing apparatus of claim 1 additionally comprising a converging section interconnecting the inner surface of the tubular body with the inner surface of the cutting-zone section.

3. The vitreous removing apparatus of claim 1 wherein a length of the cutting-zone section is less than about 0.30 inches.

4. The vitreous removing apparatus of claim 1 wherein a length of the cutting-zone section is less than about 0.20 inches.

5. The vitreous removing apparatus of claim 1 wherein a length of the cutting-zone section is about 0.10 inches.

6. The vitreous removing apparatus of claim 1 wherein a length of the distal cutting end is less than a length of the cutting-zone section.

7. The vitreous removing apparatus of claim 6 wherein a length of the distal cutting end is less than about 0.30 inches.

8. The vitreous removing apparatus of claim 6 wherein a length of the distal cutting end is less than about 0.20 inches.

9. The vitreous removing apparatus of claim 6 wherein a length of the distal cutting end is less than about 0.10 inches.

10. The vitreous removing apparatus of claim 9 wherein a length of the distal cutting end is about 0.09 inches.

11. A method for performing intraocular surgery comprising reciprocating a distal non-expanded cutting end of an inner tubular body within a reduced diameter tubular cutting-zone section of an outer tubular body.

12. The method of claim 11 additionally comprising reciprocating the distal non-expanded cutting end from about 600 to about 1600 cuts per minute.

13. The method of claim 11 additionally comprising reciprocating the distal non-expanded cutting end from about 800 to about 1600 cuts per minute.

14. The method of claim 11 additionally comprising reciprocating the distal non-expanded cutting end from about 1000 to about 1600 cuts per minute.

15. The method of claim 11 additionally comprising reciprocating the distal non-expanded cutting end from about 1200 cuts per minute to about 1600 cuts per minute.

16. The method of claim 11 additionally comprising reciprocating the distal non-expanded cutting end at about 1200 cuts per minute.
